(19) United States
(12) Patent Application Publication
(10) Pub. No.: US 2002/0107232 A1
(43) Pub. Date: Aug. 8, 2002

(54) Methods for producing sterol ester-rich compositions

(76) Inventors: Brent D. Flickinger, Decatur, IL (US); Richard Grabiel, Columbia City, IN (US); George Poppe, Forsyth, IL (US)

Correspondence Address: STERNE, KESSLER, GOLDSTEIN & FOX PLLC, 1100 NEW YORK AVENUE, N.W., SUITE 600, WASHINGTON, DC, 20005-3934, US

(21) Appl. No.: 10/034,107

(22) Filed: Jan. 3, 2002

Related U.S. Application Data
(60) Provisional application No. 60/260,918, filed on Jan. 12, 2001.

(51) Int. Cl. A61K 31/56; C07J 9/00

(52) U.S. Cl. 514/182; 552/540

(57) ABSTRACT

This invention pertains to the preparation of a sterol ester-enriched food ingredient utilizing a base-catalyzed tranesterification of free sterol with fatty acyl glyceride. Phytosterols are subject to transesterification with fatty acyl glyceride from vegetable oils in the presence of an alkali catalyst. The reaction is performed under vacuum in the range of 0.01-1 Torr. Following an initial period of transesterification, the reaction mixture is distilled to remove glycerol to enhance the formation of sterol esters. A sterol ester-rich fraction can be isolated from the reaction mixture using organic solvents in combination with aqueous washes.

### CROSS-REFERENCE TO RELATED APPLICATIONS

This non-provisional is related to U.S. provisional application No. 60/260,918, filed Jan. 12, 2001, the content of which is incorporated herein by reference.

### BACKGROUND OF THE INVENTION

1. Field of the Invention

The present invention relates to a method for the production of a sterol ester-rich composition. This invention further relates to the preparation of sterol ester-enriched food or food ingredients, dietary supplements and pharmaceutical preparations.

2. Background Art

Phytosterols are plant sterols structurally similar to cholesterol that have been known for many years to reduce cholesterol absorption and serum cholesterol levels while not being absorbed themselves. Chemically, natural sterols are C₂₆-C₃₀ steroid alcohols which have an aliphatic side chain at the C₁₇ position. The differences between a cholesterol molecule and a phytosterol molecule are primarily found in the structure of the side chain of the basic frame. Plant sterols can also be hydrogenated to produce plant stanols, i.e., phytostanols.

The use of plant sterol to lower serum cholesterol in humans has been a focus of cardiovascular research for several decades. Preparations containing mixed plant sterol as well as purified plant sterol components have demonstrated the general ability to lower serum cholesterol in humans over a range of dietary intakes. In the mid 1970s, Lilly produced the cholesterol-lowering product Cytellin® which contained between 80 and 90% beta-sitosterol.

Recently a renewed interest in the cholesterol-lowering properties of sterol has occurred through study of their hydrogenated forms known as stanols. Stanols have been shown to lower cholesterol as effectively as sterol and in some studies, stanols have demonstrated a greater ability to lower cholesterol. (Jones, P. J., MacDougall, D. E., Ntanios, F., Vanstone, C.A., “Dietary phytosterols as cholesterol-lowering agents in humans.” Can. J. Physiol. Pharmacol. 75:217-27 (1997). Stanols are produced by the hydrogenation of sterol isolated from tall oil or vegetable oils which contain beta-sitosterol as a significant proportion of total sterol compounds.

The phytosterol beta-sitosterol has also been reported to be an active ingredient in saw palmetto, reducing the severity of symptoms associated with benign prostatic hyperplasia (BPH). BPH is estimated to afflict more than fifty percent of men over the age of sixty. Approximately twenty-five percent of those afflicted require treatment. A recent German study has observed a reduction in the severity of BPH following dietary consumption of beta-sitosterol. (Berges, R. R., Windeler, J., Trampisch, H. J., Senge, T., “Randomised, placebo-controlled, double-blind clinical trial of beta-sitosterol in patients with benign prostatic hyperplasia. Beta-sitosterol Study Group.” Lancet. 345:1529-32 (1995).)

A concerted effort has been made by several companies to incorporate the healthful benefits of phytosterols into oil-based products such as margarine, cooking oils, and sprays by separately adding concentrations of phytosterol to their products. The incorporation of plant sterol and stanols into food formulations has been complicated however by the low absorption of free sterols in the gut (between 4 and 10%), their high melting temperature and the waxy texture of several phytosterols. One solution, esterification of sterol and stanols with long-chain fatty acids, improves phytosterol absorption and solubility such that the resulting phytosterol esters can be added to various food applications containing significant amounts of edible oils. Consequently, several patents describing the esterification of stanols and sterol have been assigned over the past years.

A variety of methods have previously been proposed for the production of sterol esters and sterol ester-rich ingredients to increase their solubility and absorption in the gut.

U.S. Pat. No. 3,004,043 (Stem) discloses water-soluble vegetable oil sterol derivatives, especially polyethylene glycol esters of phytosteryl acid ester compositions of dicarboxylic acids having the formula

(S)-OOCRCOO-(PEG)

wherein (S) is a phytosteryl acid ester and (PEG) is polyethylene glycol.

Patent GB 1284814 (Erickson) discloses an edible oil composition comprising a liquid glyceride base oil and a hypocholesterolemic agent such as plant sterol monocarboxylic acid ester, the acid plant sterol ester being present in an amount of from 0.5% to 10% (free sterol equivalent) by weight of the composition. Erickson discloses the derivation of the plant sterol monocarboxylic acid esters from free plant sterols by perchloric-acid-catalyzed esterification of the free sterols with monocarboxylic acid anhydrides.

Patent GB 1405346 discloses a process for the conversion of free sterols, contained in vegetable and animal oils and fats, into their corresponding fatty acid esters by transesterification in a homogeneous phase and at elevated temperature in the presence of alkali metal alcoholates or alkali metal catalysts. After washing to remove the catalyst, drying, deodorizing, and hydrogenating, the final product be used as a salad oil or mayonnaise.

U.S. Pat. No. 4,588,717 (Mitchell) disclose vitamin supplement compositions and methods of enhancing absorption of phytosterols which include the use of a fatty acid ester of a phytosterol, wherein the fatty acid forming the ester has from about 18 to 20 carbon atoms in the main carbon chain and the esterification reaction is performed at about atmospheric pressure and ambient temperature.

U.S. Pat. No. 5,502,045 (Miettinen et al.) discloses the preparation of a beta-sitostanol fatty acid ester mixture prepared by interesterifying beta-sitostanol with a fatty acid ester or containing from 2 to 22 carbon atoms in the presence of an interesterifying catalyst. A co-assigned published application, WO 98/0640 (Gylling et al.), discloses a similar beta-sitostanol fatty acid ester mixture further comprising at least 10% campestanol obtained by hydrogenation of the phytosterol mixture.

WO 99/30569 (Milstein et al.) discloses food additives useful for lowering cholesterol in humans which contains a sterol or stanol ester of a fatty acid and the formation of these fatty acid esters by reaction of a sterol or stanol and fatty acid in the presence of suitable catalyst.

U.S. Pat. No. 5,958,913 (Mittenen et al.) discloses a food composition and method for reducing the cholesterol level in the blood utilizing a 5α-saturated sterol fatty acid ester.

U.S. Pat. No. 5,892,068 (Higgins III) discloses direct esterification of stanols and sterols through the reaction of the stanol or sterol and a fatty acid using a food grade acid catalyst.

Esterification of phytosterols to fatty acids is a common practice in the art. Similarly, several patents have utilized the general esterification process involving a first step whereby fatty acyl glycerides are converted to fatty acyl methyl esters, after removal or purification of the methyl esters, a second reaction esterifies the fatty acid methyl esters with sterol or stanols to form the sterol and stanyl esters respectively. While this general technique has increased yields of the esterified products, it suffers from being commercially cumbersome since the first reaction must be driven to completion and the products separated before the second reaction be initialized. The present invention solves this problem, resulting in a more commercially viable and efficient process.

### BRIEF SUMMARY OF THE INVENTION

The present invention comprises a method for the production of a sterol ester-rich composition. The invention further relates to the use of the sterol esterrich material or an isolated sterol ester fraction as a food or as a food ingredient, beverages, nutraceuticals, dietary supplements and pharmaceuticals. Potential applications of the invention include, but are not limited to, use in lowering serum cholesterol and enhancing prostate health.

### DETAILED DESCRIPTION OF THE INVENTION

The present invention relates to a process for preparing sterol and stanol esters using a base-catalyzed transesterification of the free sterols with fatty acid glycerides coupled to removal of the produced glycerol under vacuum. According to the present invention, sterol ester-rich and purified sterol ester-rich compositions can be produced within one reaction vessel or multiple reaction vessels.

In one embodiment, the present invention relates to a method for the production of a sterol ester-rich composition comprising the steps of (a) combining a sterol composition, comprising one or more sterols, with one or more fatty acid glycerides, comprising one to three fatty acid acyl groups, to produce a blend; (b) adding an alkali catalyst to said blend to produce a reaction mixture; (c) transesterifying said reaction mixture to produce a reacted mixture; and (d) adding a food-grade acid to said reacted mixture, whereby said alkali catalyst is rendered essentially inactive, to produce said sterol ester-rich composition.

As used herein, the term “sterol” includes all phytosterols, fungal, or animal sterols, for example, sitosterol, campesterol, stigmasterol, taraxasterol, and any derivatives or reduction products of the foregoing. The term “stanol” as used herein means a hydrogenated form of a sterol. Hence, it will be appreciated that hydrogenation modifications, as well as modifications of phytosterol compounds to include, for example, small side chains, are also well within the scope of the present invention.

Any phytosterol or phytostanol which can be incorporated into an edible aqueous mixture can be utilized in the present invention. In a preferred embodiment, the phytosterol or phytostanol is selected from the group consisting of sitosterol, sitostanol, campesterol, campestanol, taraxasterol, stigmasterol, clionastanol, brassicastanol and brassicasterol, or mixtures thereof. Commercially available phytosterols are often mixtures of phytosterols that are also appropriate for use according to the present invention.

The phytosterols which are used in the present invention can be procured from a variety of natural sources. Phytosterols can be obtained from vegetable oils, vegetable oil sludge, vegetable oil distillates, and other plant oil sources such as tall oils by relatively simple and inexpensive means. For example, a preparation of sterols from vegetable oil sludge by using solvents such as methanol is taught in U.S. Pat. No. 4,420,427. Further, sitosterol can be obtained from cold pressed wheat germ oil, soy extract, or rice extract. (It will be appreciated that natural sitosterol contains about 40% alpha-sitosterol and about 60% beta-sitosterol. Both the alpha and beta forms of sitosterol can be used to form the edible phytosterol compositions of the present invention.) Stigmasterol is also found in trace amounts in cold pressed wheat germ oil, soy extract, saw palmetto and rice extract, and taraxasterol can be obtained from licorice root extract and dandelions.

Although phytostanols are found in small amounts in nature, they can easily be made from the much more abundant phytosterols by hydrogenation. Methods of preparing phytostanols from phytosterols are well-known in the art.

As used herein, the term “fatty acid glyceride” includes all glycerides such as from synthetic, plant, fungal, or animal glycerides. Fatty acid glycerides of the present invention can be present as or derived from saturated, mono-unsaturated, poly-unsaturated, or unsaturated oils or fats. It is recognized that in a preferred embodiment, these fatty acid glycerides can be present in the form of or derived from, oils such as canola, soybean, corn, sunflower, cottonseed, olive, flaxseed or NuSun sunflower or mixtures thereof.

Alkali catalysts and food-grade acids of the present invention can be any recognized by those skilled in the art. In the preferred commercially-efficient transesterification reaction method, the alkali catalyst can be selected form the group consisting of sodium methoxide and sodium ethoxide. The catalyst can be present in the reaction within the range from about 0.001 to about 5% by weight of the reaction mixture, preferably within the range from about 0.01 to about 0.7% by weight of the reaction mixture, more preferably, in a commercially efficient, transesterification reaction, the alkali catalyst is present in an amount within the range from about 0.3 to 0.5% by weight of the reaction mixture.

### Sterol Melt and Blend Production

In another embodiment, the present invention relates to combining one or more sterols combined by admixing with one or more fatty acid glycerides, to produce a blend. Alkali catalyst is added to the blend resulting in a reaction mixture. In other embodiments, the sterol composition is melted prior to combining with the glyceride(s), by heating the sterol composition to within the range from about 25° C. to about 300° C. beforehand, preferably to within the range from about 100° C. to about 200° C. beforehand, or more preferably to within the range from about 130° C. to about 180° C. beforehand. In other specific embodiments of the invention, the pressure of the reaction vessel can be adjusted to vacuum within the range of about 0.00001 to about 100 Torr, preferably to within the range of about 0.0001 and about 20 Torr, more preferably to within the range of about 0.0001 and about 5 Torr, and most preferably to within the range of about 0.0001 and about 1 Torr before, during or after or throughout the combination of the melted sterol composition with the glyceride(s).

In other specific embodiments of the invention, the blend can comprise a molar ratio of sterols to fatty acid acyl groups within the range from about 1:0.1 to about 1:20, preferably within the range from about 1:0.8 to about 1:10, or more preferably within the range from about 1:0.8 to about 1:2.

The blend can be comprised of sterol and a fatty acyl glycerol-containing oil. The blend of the present invention contains sterol, expressed as total weight of the blend, within the range from about 30% to about 90% by weight, preferably within the range from about 50% to about 70% by weight, more preferably about 58% by weight. The blend of the present invention also contains fatty acyl glycerol-containing oil, expressed as total weight of the blend, within the range from about 10% to about 70% by weight, preferably within the range from about 30% to about 50% by weight, more preferably about 42% by weight.

In further specific embodiments of the invention, the blend can be heated to a temperature to within the range from about 50° C. to about 300° C., preferably to within the range from about 120° C. to about 260° C.

### Reaction Mixture Production

The reaction mixture is typically generated by adding alkali catalyst to the sterol-fatty acid glyceride blend at elevated temperature. Preferably the temperature is adjusted to and maintained to within the range from about 50° C. to about 300° C., preferably to within the range from about 120° C. to about 260° C. during the addition of the alkali catalyst. The reaction mixture of the present invention can contain alkali catalyst in the range from about 0.01% to about 0.5% by weight, preferably 0.05 to 0.3%. In a separate embodiment, alkali catalyst can be dispersed into an amount of oil or glyceride prior to addition into the blend.

### Transesterification Reaction

Transesterification according to the present method begins upon addition of the catalyst into the blend under the defined conditions and ends when a reacted mixture has been produced. Complete (i.e. 100%) product formation is not a necessary requisite for production of a reacted mixture. In a preferred embodiment, the reaction mixture is maintained at a temperature within the range of about 50° C. to about 300° C., preferably within the range of about 120° C. to about 260° C. during the transesterification reaction; further defined in that the reaction is allowed to proceed for about 1 minute to about 24 hours, preferably about 5 minutes to about 10 hours, more preferably for about 30 minutes to about 6 hours, most preferably for about 30 minutes or about 1.5 hours.

### Neutralization

After the transesterification step, the alkali catalyst is neutralized or rendered essentially inactive by the addition of food-grade acid to the reacted mixture, thereby producing a sterol ester-rich composition. In one method according to the present invention, the reacted mixture has a temperature within the range of about 25° C. to about 200° C., preferably about 80° C. to about 100° C., during the addition of the food grade acid.

### Purification

The sterol ester-rich composition produced after neutralization above can be purified to yield glycerol and a purified sterol ester-rich composition. The purification can be performed by methods including, but not limited to, distillation, chromatography, phase separation, molecular filtration, adsorption, centrifugation, or other organic, inorganic or physical techniques as defined in the art. Distillation, for example, can be performed by transferring the sterol esterrich composition through a reaction vessel at less than atmospheric pressure, preferably within the range of about 0.01 Torr to about 1 Torr, more preferably about 0.1 Torr to about 0.5 Torr, most preferably 0.25 Torr. In an embodiment of the present invention, during distillation, the temperature is maintained within the range of about 50° C. to about 300° C., preferably at a temperature within the range from about 120° C. to about 260° C., more preferably within the range of about 140° C. to about 180° C. The rate of transfer of the sterol ester-rich composition through the reaction vessel can be constant or varied.

### Composition and Use of the Sterol Ester-Rich Composition

Useful component ranges of the sterol ester-rich composition or purified sterol ester-rich composition of the present invention include about 30-100% by weight sterol esters; about 0-25% by weight diglycerides; about 0-10% by weight monoglycerides; about 0-15% by weight sterol; and about 0-35% by weight triglycerides. These sterol ester-rich compositions can be used as foods or food ingredients such as in a dairy product, a meat product, a baked good, a nutrition bar, a confectionary product or a beverage.

Similarly, it can be seen that the sterol ester-rich compositions of the present invention can be useful in combination with a commonly-accepted pharmaceutical carrier or excipient to form a pharmaceutical preparation. When combined with an edible oil, wherein the sterol ester-rich composition comprises about 0.01-50% of the total weight, preferably about 0.1-30% of the total weight, producing a sterol ester-rich oil, it can be useful as a food or food ingredient, a medical food or medical food ingredient, or dietary supplement. Consequently, preparations of the sterol ester-rich composition, the purified sterol ester-rich composition and the sterol ester-rich oil can each be useful for either lowering serum cholesterol or effecting prostate health, in an animal subject.

Finally, the present invention allows the selection of parameters such that the fatty acid and sterols contained in the reaction mixtures can not be fully converted to fatty acid sterol esters. Therefore the preparations of the sterol esterrich composition, the purified sterol ester-rich composition and the sterol esterrich oil can contain between about 5% to about 100%, preferably about 30% to about 100% sterol esters. The sterol ester-rich reaction product contains varying degrees of unreacted starting sterol and triglyceride materials and partially-reacted triglyceride starting material which offers unique characteristics for a variety of commercial product applications.

Having now generally described the invention, the same will be more readily understood through reference to the following Examples which are provided by way of illustration, and are not intended to be limiting of the present invention, unless specified.

### EXAMPLES

### Example 1

Prilled sterol (700 g) were melted then heated to 160° C. under vacuum (0.25 Torr) and with stirring. After 30 minutes, canola salad oil (500 g) was added then allowed to stir under vacuum (0.25 Torr) until a temperature of 160° C. was maintained. Sodium methoxide (0.3%) was added quickly. The reaction was allowed to proceed under vacuum (0.25 Torr) at 160° C. for 30 minutes. The reaction mixture was then passed through a pilot plant scale oil deodorizer with the feed tube temperature of 150° C. and column temperature of 170° C. under vacuum (0.25 Torr).

### Example 2

A 60 g amount of esterification reaction mixture described in Example 1 was dissolved in 300 ml of n-heptane. Chilled water (100 ml) was added to the organic phase. The phases were agitated by gentle rocking then remained undisturbed for 15 minutes. The aqueous phase was decanted and the aqueous wash repeated. Following removal of the second aqueous wash, the organic phase was filtered to remove precipitated free sterol. The organic phase was filtered through anhydrous magnesium sulfate followed by removal of the n-heptane using a rotary evaporator.

### Example 3

Prilled sterol (3000 g) were melted at 170° C. then degassed under vacuum (400-500 mTorr) for 30 minutes. Heated, degassed canola oil (2100 g) was added to the molten sterols. Sodium methoxide (16.5 g) dispersed in canola oil (150 g) was added to the reaction mixture under vigorous stirring. The reaction mixture was recirculated through a molecular distillation unit (MDU) (feed temperature 170° C.; MDU temperature 90° C.; MDU wiper speed 200 rpm) for 1.5 hours to generate a sterol-ester rich fraction.

### Example 4

A mixture of 9 g soybean salad oil and 2 g sterol-ester rich fraction (from the esterification of prilled sterol using canola oil fatty acyl glyceride) was prepared. The mixture was combined under mild heating and gentle stirring. After prolonged refrigeration at 5° C., no visible precipitation of components from the oil-sterol ester mixture resulted.

All publications mentioned herein are hereby incorporated in their entirety by reference. Further, in view of the foregoing description taken with the examples, those skilled in the art should be able to practice the invention in various enablements without departing from the spirit and scope of the invention as defined in the claims.

What is claimed is:

1. A method for the production of a sterol ester-rich composition comprising the steps of:
(a) combining a sterol composition, comprising one or more sterols, with one or more fatty acid glycerides, comprising one to three fatty acid acyl groups, to produce a blend;
(b) adding an alkali catalyst to said blend to produce a reaction mixture;
(c) transesterifying said reaction mixture to produce a reacted mixture; and
(d) adding a food-grade acid to said reacted mixture, whereby said alkali catalyst is rendered essentially inactive, to produce said sterol esterrich composition.

2. The method of claim 1, wherein at step (a) said sterol composition is melted prior to combining with said one or more fatty acid glycerides.

3. The method of claim 1, additionally comprising the step of (e) purifying the product of step (d) to produce glycerol and a purified sterol esterrich composition.

4. The method of claim 3, wherein the product of step (d) is purified by distillation to produce glycerol and said purified sterol ester-rich composition.

5. The method of claim 1, wherein said blend comprises (a) said sterols and (b) said fatty acid acyl groups, wherein the molar ratio of (a) to (b) is between 1:0.8 and 1:10.

6. The method of claim 1, wherein said blend comprises from about 30% to about 90% by weight of said sterols.

7. The method of claim 1, wherein said blend comprises from about 50% to about 70% by weight of said sterols.

8. The method of claim 1, wherein said blend comprises from about 58% by weight of said sterols.

9. The method of claim 5, wherein the molar ratio of (a) to (b) is between 1:0.8 and 1:2.

10. The method of claim 2, wherein said sterol composition is melted by heating to a temperature between 130° C. and 180° C. and is placed under a vacuum between 0.0001 Torr and 20 Torr.

11. The method of claim 7, wherein said vacuum is between 0.0001 Torr and 5 Torr.

12. The method of claim 8, wherein said vacuum is between 0.0001 Torr and 1 Torr.

13. The method of claim 1, wherein said fatty acid glycerides are selected from the group consisting of canola, soybean, corn, sunflower, cottonseed, olive and flaxseed fatty acid glycerides.

14. The method of claim 1, wherein said alkali catalyst is selected from the group consisting of sodium methoxide and sodium ethoxide.

15. The method of claim 1, wherein at step (a) said blend is heated to a temperature between 120° C. and 260° C.

16. The method of claim 1, wherein at step (b) said blend is maintained at a temperature between 120° C. and 260° C. during said adding of said alkali catalyst.

17. The method of claim 1, wherein said alkali catalyst comprises 0.01-0.5% by weight of said reaction mixture.

18. The method of claim 14, wherein said alkali catalyst comprises 0.05-0.3% by weight of said reaction mixture.

19. The method of claim 1, wherein said alkali catalyst is dispersed in a fatty acid glyceride prior to adding in step (b).

20. The method of claim 1, wherein at step (c) said reaction mixture is maintained at a temperature between 120° C. and 260° C. during said transesterifying.

21. The process of claim 1, wherein at step (c) said transesterifying proceeds for 0.1 to 10 hours.

22. The process of claim 18, wherein said transesterifying proceeds for 0.5 to 6 hours.

23. The process of claim 1, wherein at step (d) said reacted mixture has a temperature between 80° C. and 100° C. during said adding of said food-grade acid.

24. The process of claim 1, additionally comprising the step of:
(e) transferring said sterol ester-rich composition through a reaction vessel at less than atmospheric pressure to distill said sterol ester-rich composition producing glycerol and a purified sterol ester-rich composition.

25. The process of claim 21, wherein at step (e) the temperature of said sterol ester-rich composition is maintained at a temperature between 120° C. and 260° C. during distillation.

26. The process of claim 21, wherein at step (e) the rate of said transferring is not constant.

27. A food or food ingredient comprising the composition of claim 24.

28. A dietary supplement comprising the composition of claim 24.

29. A pharmaceutical preparation comprising the composition of claim 24 and a pharmaceutically acceptable carrier.

30. A composition produced by the process of claim 1.

31. A composition produced by the process of claim 3.

32. A composition produced by the process of claim 21.
